(19) United States
(12) Patent Application Publication
(10) Pub. No.: US 2026/0117068 A1
(43) Pub. Date: Apr. 30, 2026

(54) PARTICLE HYDROGEL WITH EXCELLENT MECHANICAL PROPERTIES, AND PREPARATION METHOD AND USE THEREOF

(71) Applicant: Hefei University of Technology, Hefei (CN)

(72) Inventors: Yang LU, Hefei (CN); Chaowei LU, Hefei (CN); Xu YAN, Hefei (CN); Huai YANG, Hefei (CN); Xi JING, Hefei (CN); Haojie LU, Hefei (CN); Yonghong SONG, Hefei (CN)

(73) Assignee: Hefei University of Technology, Hefei (CN)

(21) Appl. No.: 19/433,314

(22) Filed: Dec. 26, 2025

(30) Foreign Application Priority Data
Dec. 30, 2024 (CN) 202411964791.X

(51) Int. Cl. C08L 89/00; A61L 24/00; C08K 3/22; C08K 3/26

(52) U.S. Cl. CPC C08L 89/00; A61L 24/0031; A61L 24/0089; C08K 3/22; C08K 2003/2275; C08K 2003/265; C08K 2201/011

(57) ABSTRACT

A preparation method of a particle hydrogel includes: mixing a gelatin nanoparticle, a calcium carbonate nanoparticle, an iron oxide nanoparticle, and a sodium alginate solution under alkaline conditions, and adding GDL to produce the particle hydrogel with improved mechanical properties, which is abbreviated as Ca-Alg/MCG. The present disclosure endows the hydrogel system with properties such as catheter injectability, vascular deliverability, and intravascular hardenability. The particle hydrogel can be delivered to fine blood vessels of tumors. The mechanical properties of the particle hydrogel can be gradually enhanced over time, which further improves the embolization efficacy of the particle hydrogel in blood vessels. Magnetic nanoparticles endow the particle hydrogel with magnetothermal responsiveness. Thus, the particle hydrogel can undergo a rapid temperature rise under an alternating magnetic field to further kill tumor cells. The particle hydrogel demonstrates improved embolization efficacy in rabbit renal embolization models and rabbit liver cancer-embolization models.

### CROSS-REFERENCE TO RELATED APPLICATIONS

This application claims priority to Chinese Patent Application No. 202411964791.X with a filing date of Dec. 30, 2024. The content of the aforementioned application, including any intervening amendments thereto, is incorporated herein by reference.

### TECHNICAL FIELD

The present disclosure relates to the technical field of preparation of composite materials, and specifically relates to a particle hydrogel with improved mechanical properties, and a preparation method and use thereof.

### BACKGROUND

Hepatocellular carcinoma (HCC) is the most common malignant liver tumor. HCC cases account for 85% to 90% of all liver cancer cases. Approximately 700,000 to 800,000 new HCC cases are reported globally each year. Transcatheter arterial embolization (TAE) is a mainstream treatment strategy. With TAE, an embolic agent can be selectively injected into tumor-feeding arteries to block the blood supply to a tumor and suppress the progression of the tumor. TAE can significantly extend the survival time of patients with intermediate-stage to advanced-stage HCC. Traditional embolic agents can be classified into solid embolic agents (such as polyvinyl alcohol microspheres and gelatin sponge particles) and liquid embolic agents (such as lipiodol). However, due to limited sizes, the solid embolic agents cannot be filled into fine or irregular blood vessels. Additionally, the solid embolic agents tend to aggregate, which may lead to catheter blockage. In contrast, the liquid embolic agents are well suited for irregular target blood vessels. Lipiodol demonstrates improved fluidity and strong vascular conformability, and can rapidly enter the peripheral arteries of tumors. However, due to a relatively low mechanical strength, lipiodol can easily be cleared by blood flow, potentially resulting in vascular recanalization within tumor tissues. Therefore, enhancing both the delivery characteristics of an embolic agent in blood vessels and the mechanical properties of the embolic agent is key to addressing the aforementioned issues.

In recent years, shear-thinning hydrogels have garnered significant attention in the field of embolic agents for blood vessels. This is because shear-thinning hydrogels exhibit fluid-like behaviors in catheters when being injected and can return to the original gel state after the applied stress is removed. Particle hydrogels are a type of shear-thinning hydrogels. In particle hydrogels, nanoparticles, as assembly units, are subjected to bottom-up assembly through non-covalent interactions to form a three-dimensional network structure. When a network of a particle hydrogel undergoes an external stress or a shearing action, a viscosity of the particle hydrogel decreases sharply, resulting in fluidity. Once the external force is removed, the original viscosity can be recovered quickly, indicating structural self-repairing capability. Due to unique rheological properties, improved biosafety, injectability, and catheter deliverability, particle hydrogels are regarded as highly promising novel embolic materials for blood vessels. However, such a particle hydrogel network formed through physical non-covalent interactions shows poor mechanical properties, and can hardly withstand a significant external load. Consequently, after being implanted into arterial vessels, such a particle hydrogel network is easily destructed under the shearing and impacting of high-velocity blood flow, making it challenging to achieve stable and durable embolization for the arterial vessels. Chemical cross-linking can effectively improve the mechanical properties and structural stability of particle hydrogels. However, this technical approach often negatively impacts the injectability of particle hydrogels, and the corresponding particle hydrogels cannot be delivered through catheters into fine blood vessels of tumors. Thus, this technical approach fails to meet the requirements of clinical embolization procedures. Further, chemical cross-linking agents have potential biotoxicity, which limits the application of chemical cross-linking agents in clinical procedures. Therefore, the development of colloidal hydrogel-based embolic agents for liver cancer that possess prominent mechanical properties, injectability, deliverability, and biosafety still faces difficulties and challenges.

Through long-term research and practices of the inventors, the present disclosure is provided to overcome the above defects.

### SUMMARY OF PRESENT INVENTION

An objective of the present disclosure is to address the issue of how to improve the mechanical properties of a particle hydrogel without compromising the injectability of the particle hydrogel, and to provide a particle hydrogel with improved mechanical properties, and a preparation method and use thereof.

To achieve the above objective, the present disclosure provides a preparation method of a particle hydrogel with improved mechanical properties, including the following steps:
S1, mixing a gelatin nanoparticle, an iron oxide nanoparticle, a calcium carbonate nanoparticle, and sodium alginate under alkaline conditions; and
S2, adding a glucono-δ-lactone (GDL) powder for pH regulation to produce the particle hydrogel with improved mechanical properties.

The hydrolysis of GDL causes a gradual decrease in pH. During this process, the gelatin nanoparticle transitions from a negatively charged state to a positively charged state, and the iron oxide nanoparticle still remains negatively charged. As a result, an electrostatic interaction is generated between the gelatin nanoparticle and the iron oxide nanoparticle. Subsequently, the calcium carbonate nanoparticle undergoes acidolysis by hydrogen ions resulting from the hydrolysis of GDL to release calcium ions. These calcium ions are cross-linked with sodium alginate to further enhance the mechanical properties of the particle hydrogel produced based on the aforementioned electrostatic interaction. In this way, the particle hydrogel with improved mechanical properties can be acquired.

In the S1, a preparation process of the gelatin nanoparticle includes the following steps:
S111, dissolving a gelatin in deionized water under heating to produce a gelatin solution; adding acetone to the gelatin solution, and allowing to stand at room temperature for 1 h to produce a supernatant and a precipitate; discarding the supernatant; and dissolving the precipitate in deionized water, and conducting lyophilization to produce a lyophilized high-molecular-weight gelatin; and
S112, dissolving the lyophilized high-molecular-weight gelatin obtained in the S111 in deionized water, and adjusting a pH with dilute hydrochloric acid to 2.5; under stirring, adding acetone dropwise to produce a gelatin dispersion; adding a glutaraldehyde solution to the gelatin dispersion at room temperature, and stirring for 16 h in the dark; adding an aqueous glycine solution, and stirring for 1 h; and conducting filtration and centrifugal washing to produce the gelatin nanoparticle.

In the S1, a preparation process of the iron oxide nanoparticle includes the following steps:
S121, adding 1.08 g of ferric chloride hexahydrate to a mixture of 10 mL of ethylene glycol and 30 mL of diethylene glycol, and stirring for 30 min to produce a first mixed solution;
S122, adding 0.1 g of polyacrylic acid to the first mixed solution obtained in the S121, and stirring for 30 min to produce a second mixed solution;
S123, adding 6 g of anhydrous sodium acetate to the second mixed solution obtained in the S122, and stirring for 1 h to produce a third mixed solution; and
S124, adding the third mixed solution obtained in the S123 to a high-pressure reactor, and conducting a reaction at a high temperature to produce a black product; and subjecting the black product to centrifugal washing to produce the iron oxide nanoparticle.

In the S124, the reaction is conducted at 200° C. for 12 h.

In the S1, a preparation process of the calcium carbonate nanoparticle includes the following steps:
S131, dissolving 150 mg of calcium chloride dihydrate in 100 mL of ethanol to produce a calcium chloride solution;
S132, placing 5 g of an ammonium bicarbonate powder in a sealed container; and
S133, adding the calcium chloride solution obtained in the S131 to the sealed container in the S132; placing the sealed container in an oven, and conducting a reaction under vacuum to produce a white product; and subjecting the white product to centrifugal washing to produce the calcium carbonate nanoparticle.

In the S133, the reaction is conducted at 40° C. for 24 h.

In the S1, the gelatin nanoparticle, the iron oxide nanoparticle, the calcium carbonate nanoparticle, and the sodium alginate are mixed under vortexing at a pH of about 11.

In the S2, a solid content in the particle hydrogel with improved mechanical properties is 11.0 w/v % to 13.5 w/v %.

The present disclosure also discloses a use of a particle hydrogel with improved mechanical properties produced by the preparation method described above in preparation of an interventional embolic agent for embolization/magnetic hyperthermia combined therapy of liver cancer.

In the particle hydrogel system including a gelatin nanoparticle and an iron oxide nanoparticle provided by the present disclosure, sodium alginate and a calcium carbonate nanoparticle are introduced. In an environment of gradual hydrolysis of GDL, the mechanical properties of the particle hydrogel system can be progressively enhanced. Thus, the present disclosure develops a particle hydrogel with improved mechanical properties, which is abbreviated as Ca-Alg/MCG. Sodium alginate is a natural polyanionic polysaccharide molecule with a strong affinity for divalent ions (such as calcium ions). Divalent ions can bind to α-L-guluronic acid in alginate molecules to form ionic cross-linking. Calcium ions are gradually dissociated from calcium carbonate and chelate with sodium alginate, thereby progressively enhancing the mechanical properties of the particle hydrogel. A storage modulus of the particle hydrogel can increase to 11 times the original storage modulus after two hours. The particle hydrogel demonstrates improved embolization efficacy in both rabbit renal embolization models and rabbit liver cancer-embolization models. Additionally, due to the responsiveness of magnetic nanoparticles to an alternating magnetic field, the particle hydrogel can generate heat to further kill tumor cells.

Compared with the prior art, the present disclosure has the following beneficial effects:
1. The particle hydrogel prepared by the present disclosure is produced as follows: A three-dimensional hydrogel network is first formed through the controllable electrostatic assembly of building units. Then, sodium alginate, a natural polysaccharide, chelates with calcium ions to enhance the mechanical properties of the particle hydrogel. This preparation process does not involve complex chemical cross-linking or toxic cross-linking agents. Both the synthetic raw materials and the cross-linking mode exhibit improved biocompatibility, which is conducive to clinical translation.
2. The particle hydrogel with improved mechanical properties prepared in the present disclosure has both injectability and mechanical properties enhanced. Particle hydrogels generally have inherently weak mechanical properties, which limits the application of particle hydrogels in arterial vessels. A common approach to improve the mechanical properties of hydrogels involves increasing a cross-linking density of a hydrogel through irreversible covalent bonds. However, this approach typically results in reduced injectability of a hydrogel. Therefore, the particle hydrogel with improved mechanical properties prepared in the present disclosure exhibits improved injectability when initially prepared, and possesses mechanical properties that can be further improved over time.
3. The particle hydrogel with improved mechanical properties prepared in the present disclosure exhibits improved magnetic field responsiveness, and can undergo a rapid temperature rise under an alternating magnetic field to further kill tumor cells. Thus, the particle hydrogel can achieve the synergistic therapy of embolization and magnetic hyperthermia.

### BRIEF DESCRIPTION OF THE DRAWINGS

FIG. 1 shows a scanning electron microscopy image (A) and a hydrodynamic particle size distribution (B) of a gelatin nanoparticle;

FIG. 2 shows a scanning electron microscopy image (A) and a hydrodynamic particle size distribution (B) of an iron oxide nanoparticle;

FIG. 3 shows a scanning electron microscopy image (A) and a hydrodynamic particle size distribution (B) of a calcium carbonate nanoparticle;

FIG. 4 shows scanning electron microscopy images (A) of an internal structure of MCG and scanning electron microscopy images (B) of an internal structure of Ca-Alg/MCG;

FIG. 5 shows pictures for molding and demolding of MCG and Ca-Alg/MCG;

FIG. 6 shows deformation conditions of MCG and Ca-Alg/MCG under an external load;

FIG. 7 shows injection forces for Ca₀-Alg₁/MCG, Ca₀-Alg/MCG, and Ca₀-Alg₂/MCG to pass through a 2.6 F catheter;

FIG. 8 shows embolization pressures of Ca₀-Alg₁/MCG, Ca₀-Alg/MCG, and Ca₀-Alg₂/MCG;

FIG. 9 shows the comparison of storage moduli of MCG, Ca₀-Alg/MCG, Ca_{0.5}-Alg/MCG, Ca_{1.0}-Alg/MCG, and Ca_{1.5}-Alg/MCG before and after gelation;

FIG. 10 shows shear-thinning test results for freshly prepared Ca₀-Alg/MCG, Ca_{0.5}-Alg/MCG, Ca_{1.0}-Alg/MCG, and Ca_{1.5}-Alg/MCG;

FIG. 11 shows injection forces for freshly prepared Ca₀-Alg/MCG, Ca_{0.5}-Alg/MCG, Ca_{1.0}-Alg/MCG, and Ca_{1.5}-Alg/MCG to pass through catheters;

FIG. 12 shows peak retention test results for freshly prepared Ca_{1.0}-Alg/MCG;

FIG. 13 shows changes of moduli of MCG and Ca_{1.0}-Alg/MCG over time;

FIG. 14 shows a change curve of a cumulative released quantity of calcium ions over time;

FIG. 15 shows embolization pressures of MCG and Ca_{1.0}-Alg/MCG;

FIG. 16 shows digital subtraction angiography (DSA) images and an X-ray image of Ca_{1.0}-Alg/MCG in a rabbit renal embolization model;

FIG. 17 shows computed tomography (CT) images of a blank control group and a 28 d post-embolization group;

FIG. 18 shows color Doppler ultrasound (CDU) images and contrast-enhanced ultrasound (CEUS) images of the blank control group and the 28 d post-embolization group;

FIG. 19 shows three-dimensional renal reconstruction images for the blank control group and the 28 d post-embolization group;

FIG. 20 shows pictures of kidneys from the blank control group and the 28 d post-embolization group;

FIG. 21 shows hematoxylin and eosin (H&E) staining images of renal tissues on day 0 and day 28 after embolization;

FIG. 22 shows Prussian blue (PB) staining images of renal tissues on day 0 and day 28 after embolization;

FIG. 23 shows a magnetothermal heating effect of Ca_{1.0}-Alg/MCG in a tumor model;

FIG. 24 shows X-ray images and DSA images of a tumor in a liver cancer rabbit before and after vascular embolization;

FIG. 25 shows pictures for liver cancer rabbits in an untreated group, a TAE therapy group, and a TAE/magnetic hyperthermia combined therapy group on day 14 after a treatment;

FIG. 26 shows H&E staining images for the liver cancer rabbits in the untreated group, the TAE therapy group, and the TAE/magnetic hyperthermia combined therapy group on day 14 after a treatment; and

FIG. 27 shows PB staining images for the liver cancer rabbits in the untreated group, the TAE therapy group, and the TAE/magnetic hyperthermia combined therapy group on day 14 after a treatment.

### DETAILED DESCRIPTION OF THE EMBODIMENTS

The above and other technical features and advantages of the present disclosure will be described in further detail below in conjunction with the accompanying drawings.

### Example 1

### 1. Preparation of a Gelatin Nanoparticle

A spherical gelatin nanoparticle was prepared by a two-step desolvation process, including the following steps:
(1) 25 g of a gelatin was dissolved in 500 mL of deionized water to produce a gelatin solution. Then, 500 mL of acetone was added to the gelatin solution to produce a mixed solution. The mixed solution was allowed to stand at room temperature for 1 h to produce a supernatant and a gelatin precipitate. The supernatant was discarded. The gelatin precipitate was dissolved in deionized water at 50° C. Then, lyophilization was conducted to produce a high-molecular-weight gelatin.
(2) 3.75 g of the high-molecular-weight gelatin was dissolved in 75 mL of deionized water, and a pH was adjusted with dilute hydrochloric acid to 2.5. Under stirring at 1,000 rpm, 225 mL of acetone was added by a syringe pump at a rate of 10 mL/min dropwise to a gelatin solution. A resulting reaction solution was cooled to room temperature, and 550 μL of a glutaraldehyde solution (25 wt %) was then added. Stirring was conducted for 16 h at 600 rpm in the dark to produce a gelatin dispersion.
(3) 100 mL of a 300 mM aqueous glycine solution was added to the gelatin dispersion obtained in the step (2) to remove the unreacted aldehyde groups. Stirring was conducted for 1 h, and a resulting mixture was filtered through a filter sieve with a pore size of 100 μm to obtain a filter cake. The filter cake was then subjected to centrifugal washing, and dispersed in an acetone-water solution (a volume ratio of acetone to water was 1:3) for later use.

### 2. Preparation of a Ferroferric Oxide Nanoparticle

The ferroferric oxide nanoparticle was prepared by a solvothermal process, including the following steps:
(1) 1.08 g of ferric chloride hexahydrate was dissolved in a mixture of 10 mL of ethylene glycol and 30 mL of diethylene glycol, and stirring was conducted for 30 min to allow complete dissolution. Then, 0.1 g of polyacrylic acid was added, and stirring was conducted for 30 min. Finally, 6 g of anhydrous sodium acetate was added, and stirring was conducted for 1 h to produce a mixed solution.
(2) The mixed solution obtained in the step (1) was transferred to a high-pressure reactor, and a reaction was conducted at 200° C. for 12 h.
(3) After the reaction was completed, a resulting black product was subjected to repeated centrifugal washing for later use.

### 3. Preparation of a Calcium Carbonate Nanoparticle

The calcium carbonate nanoparticle was prepared by a gas diffusion process, including the following steps:
(1) 150 mg of calcium chloride dihydrate was dissolved in 100 mL of ethanol to produce a calcium chloride solution. The calcium chloride solution was placed in a sealed container.
(2) 5 g of ammonium bicarbonate was added to the sealed container.
(3) The sealed container was placed in an oven at 40° C., and a reaction was conducted under vacuum for 24 h. After the reaction was completed, a resulting white product was subjected to centrifugal washing for later use.

### 4. Preparation of a Particle Hydrogel With Enhanced Mechanical Properties, Including the Following Steps:

The prepared alkaline (pH=11) gelatin nanoparticle dispersion, iron oxide nanoparticle dispersion, sodium alginate solution, and calcium carbonate nanoparticle dispersion were thoroughly mixed under vortexing to produce a system in which final concentrations of the gelatin nanoparticle, the iron oxide nanoparticle, the sodium alginate, and the calcium carbonate were 8 w/v %, 2 w/v %, 2 w/v %, and 1 w/v %, respectively. Then, 50 mg of a GDL powder was added to the system for pH regulation to induce gelation, so as to produce the particle hydrogel Ca_{1.0}-Alg/MCG with enhanced mechanical properties.

### Example 2

Preparation conditions in this example were the same as those in Example 1, except that a content of the calcium carbonate nanoparticle was 0.5 w/v %, an amount of the GDL powder added was 32 mg, and a product was abbreviated as Ca_{0.5}-Alg/MCG.

### Example 3

Preparation conditions in this example were the same as those in Example 1, except that a content of the calcium carbonate nanoparticle was 1.5 w/v %, an amount of the GDL powder added was 67 mg, and a product was abbreviated as Ca_{1.5}-Alg/MCG.

### Example 4

Preparation conditions in this example were the same as those in Example 1, except that a content of the calcium carbonate nanoparticle was 0, a content of the sodium alginate was 2 w/v %, an amount of the GDL powder added was 14 mg, and a product was abbreviated as Ca₀-Alg/MCG.

### Example 5

Preparation conditions in this example were the same as those in Example 1, except that a content of the calcium carbonate nanoparticle was 0, a content of the sodium alginate was 1 w/v %, an amount of the GDL powder added was 14 mg, and a product was abbreviated as Ca₀-Alg₁/MCG.

### Example 6

Preparation conditions in this example were the same as those in Example 1, except that a content of the calcium carbonate nanoparticle was 0, a content of the sodium alginate was 3 w/v %, an amount of the GDL powder added was 14 mg, and a product was abbreviated as Ca₀-Alg₂/MCG.

### Comparative Example 1

The prepared alkaline (pH: about 11) gelatin nanoparticle dispersion and iron oxide nanoparticle dispersion were thoroughly mixed under vortexing to produce a system in which final concentrations of the gelatin nanoparticle and the iron oxide nanoparticle were 8 w/v % and 2 w/v %, respectively. Then, 14 mg of a GDL powder was added to the system for pH regulation to induce gelation, so as to produce a particle hydrogel with improved mechanical properties, which was abbreviated as MCG.

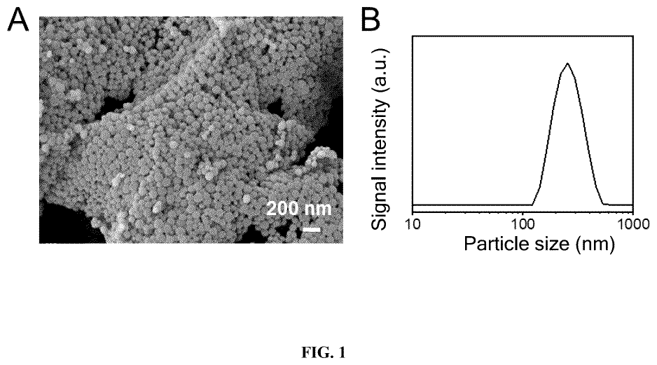

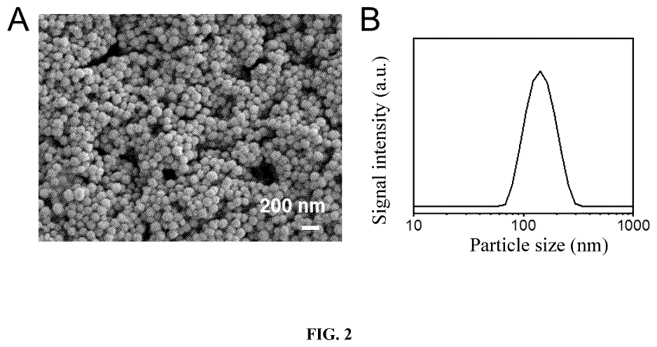

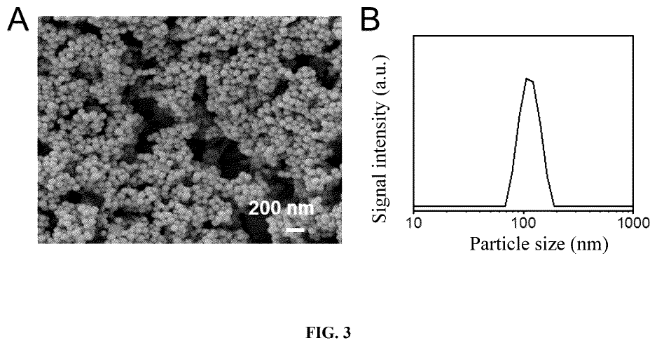

FIG. 1 shows a scanning electron microscopy image and a hydrodynamic particle size distribution of a gelatin nanoparticle. The gelatin nanoparticle presents a uniform and smooth surface and has a particle size of approximately 240 nm. FIG. 2 shows a scanning electron microscopy image and a hydrodynamic particle size distribution of an iron oxide nanoparticle. The iron oxide nanoparticle is a rough spherical particle with a particle size of approximately 130 nm. FIG. 3 shows a scanning electron microscopy image and a hydrodynamic particle size distribution of a calcium carbonate nanoparticle. The calcium carbonate nanoparticle has a particle size of approximately 110 nm.

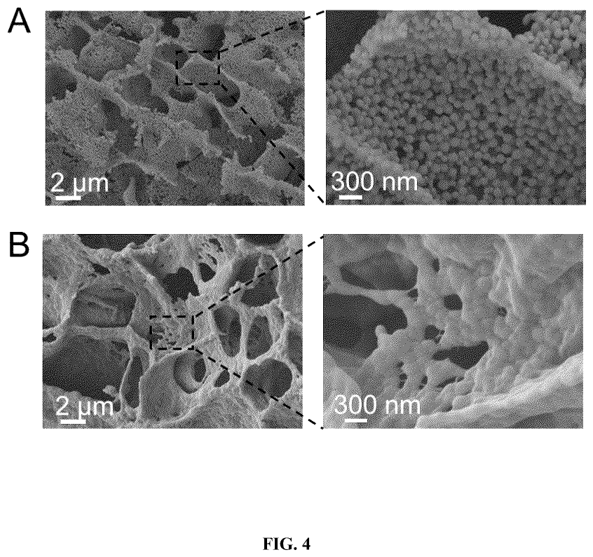

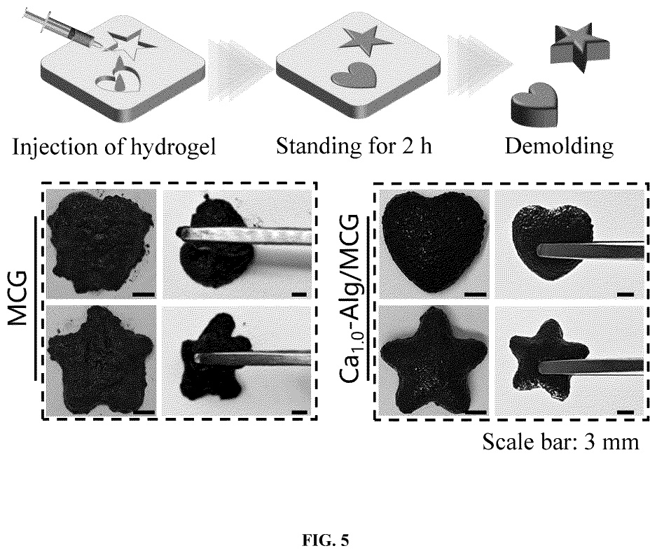

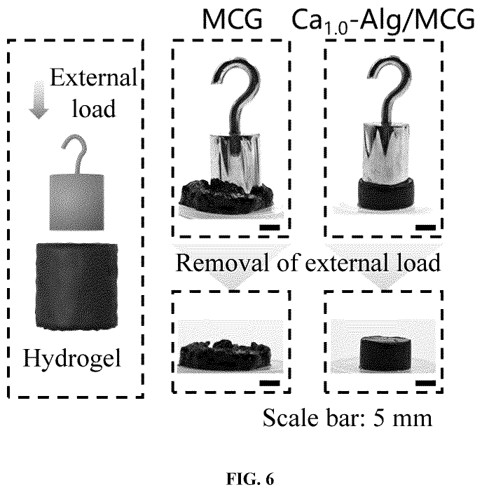

As shown in FIG. 4, internal structures of a particle hydrogel produced after gelation (MCG) and a particle hydrogel with improved mechanical properties (Ca-Alg/MCG) were compared. According to comparison results, MCG merely has a three-dimensional network assembled from particles, but Ca-Alg/MCG exhibits a polymer network encapsulating nanoparticles. As shown in FIG. 5, shapes of a particle hydrogel (MCG) and a particle hydrogel with improved mechanical properties (Ca-Alg/MCG) after molding and demolding were compared. Compared to MCG, Ca-Alg/MCG shows a fine contour and can be lifted with tweezers after demolding, indicating superior mechanical properties. As shown in FIG. 6, Ca-Alg/MCG can withstand an external force and is not prone to deformation. Moreover, Ca-Alg/MCG can restore to the original shape upon removal of an external force, and demonstrates stronger mechanical properties than MCG.

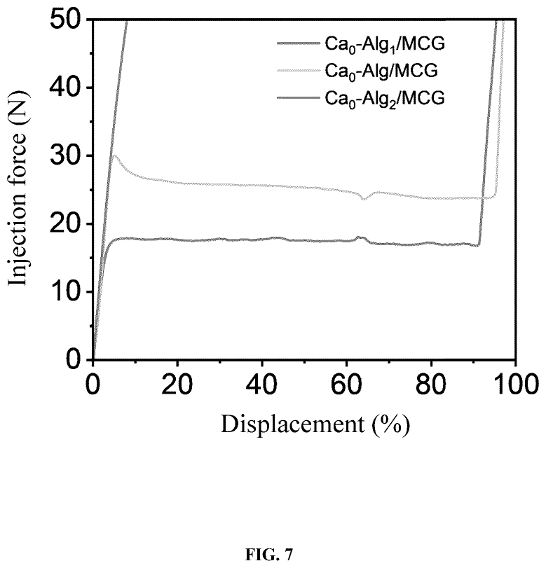

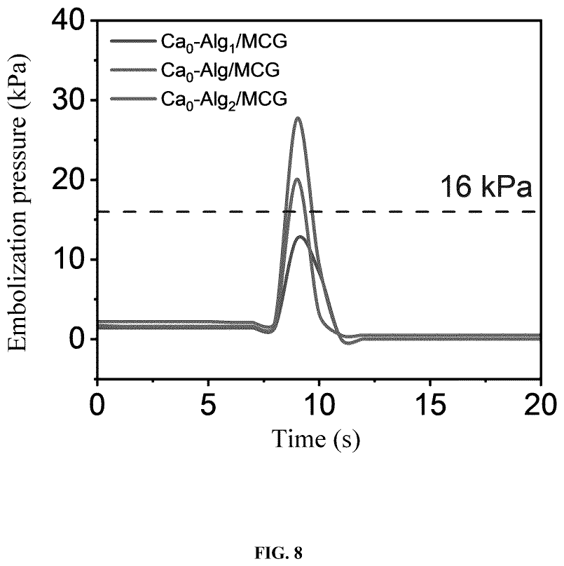

To clarify the influence of a content of sodium alginate on the mechanical properties of the particle hydrogel, sodium alginate was added at different contents to the particle hydrogel MCG. As shown in FIG. 7, when the content of sodium alginate was 3 w/v %, a force required for the particle hydrogel to pass through a 2.6 F catheter at an injection rate of 1 mL/min exceeded 50 N, resulting in non-injectability. When the content of sodium alginate was 1 w/v % or 2 w/v %, an injection force remained below 50 N, enabling easy injection through a catheter. As shown in FIG. 8, embolization pressures under these three different sodium alginate contents were tested. When the content of sodium alginate was 1 w/v %, a corresponding embolization pressure was lower than a physiological arterial pressure of the human (16 kPa). However, embolization pressures under the other two sodium alginate contents both exceeded 16 kPa, indicating a specified ability to withstand blood flow impact.

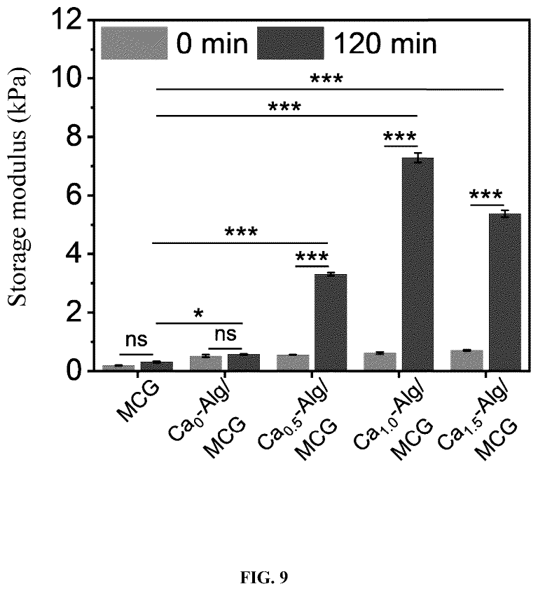

As shown in FIG. 9, storage moduli of hydrogels in Examples 1, 2, 3, and 4 and Comparative Example 1 before and after gelation were compared. In Comparative Example 1, after the gelation of MCG was induced by electrostatic assembly, a storage modulus of MCG increased from 192.2±11.0 Pa to 312.4±27.2 Pa. In Example 4, a storage modulus of Ca₀-Alg/MCG after gelation was 573.3±15.6 Pa, indicating that the gelation did not significantly affect a storage modulus of MCG. In Example 1, a storage modulus of Ca_{1.0}-Alg/MCG increased from 615.7±39.8 Pa before gelation to 7,284.5±164.2 Pa after gelation, indicating improved mechanical properties. In Example 2, Ca_{0.5}-Alg/MCG after gelation had a relatively low storage modulus of 3,306.4±56.9 Pa. This is because a low calcium carbonate nanoparticle content ultimately leads to an insufficient degree of cross-linking. In Example 3, a storage modulus of Ca_{1.5}-Alg/MCG after gelation was 5,376.7±119.7 Pa. In this example, a calcium carbonate nanoparticle content was too high. The excessive calcium ions underwent non-uniform chelation with sodium alginate, which hindered the formation of a symmetric egg-box dimer and caused the cross-linking both between and inside alginate molecules. Ultimately, a hardness of the hydrogel was reduced. According to test results, the mechanical properties of the hydrogel of Example 1 were most significantly improved.

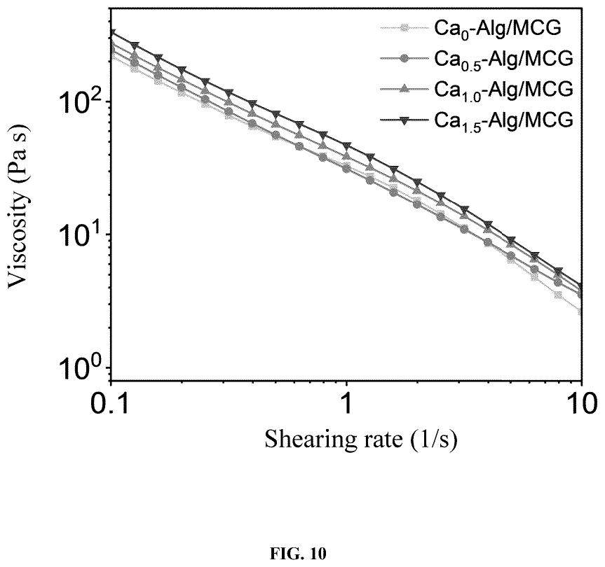

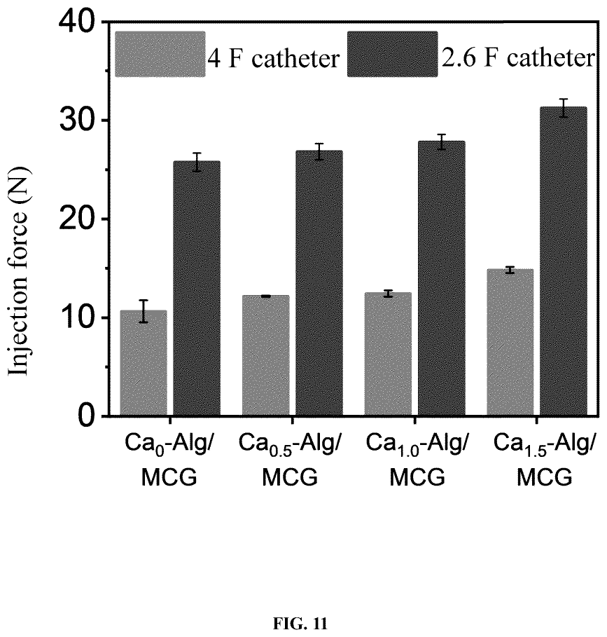

As shown in FIG. 10, shear-thinning characteristics of the hydrogels freshly prepared in Examples 1, 2, 3, and 4 were tested. A viscosity of the hydrogel decreased with the increase in shearing rate. As shown in FIG. 11, the hydrogels freshly prepared in Examples 1, 2, 3, and 4 were tested for catheter injectability.

Injection forces for these four hydrogels to pass through a 4 F catheter were 12.4±0.3 N, 12.2±0.1 N, 14.8±0.3 N, and 10.6±1.1 N, respectively. Injection forces for these four hydrogels to pass through a 2.6 F catheter were 27.8±0.7 N, 26.8±0.8 N, 31.2±0.9 N, and 25.8±0.9 N, respectively. The results indicate that the hydrogels freshly prepared in Examples 1, 2, 3, and 4 all possess prominent catheter injectability.

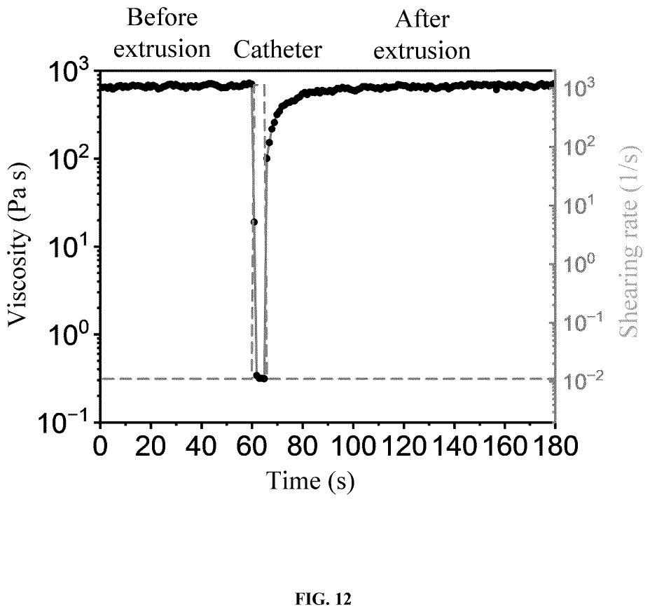

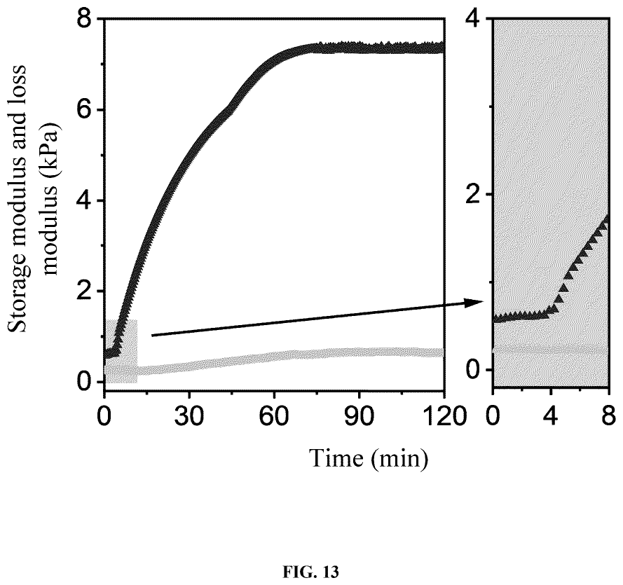

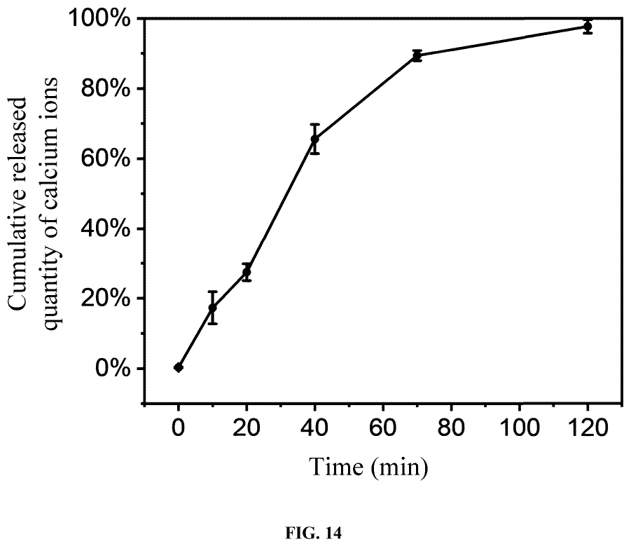

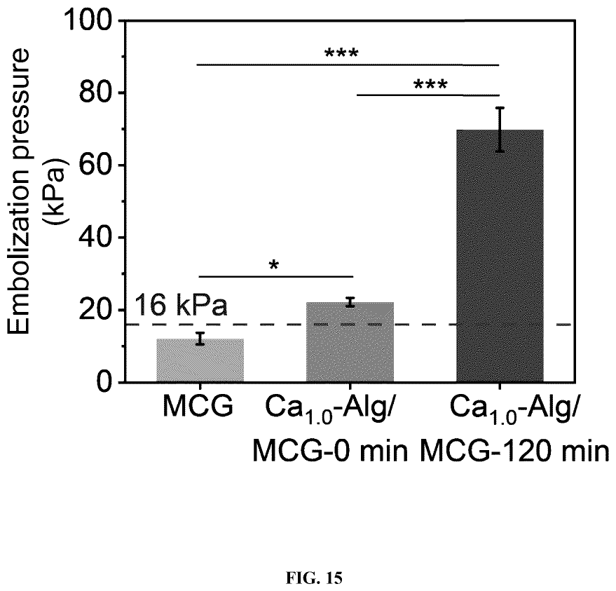

To evaluate a viscosity change of a hydrogel during an extrusion process through a catheter, a peak retention test was conducted. As shown in FIG. 12, at the beginning of the extrusion process, the hydrogel was subjected to a low shearing rate (0.01 s⁻¹). Subsequently, when the hydrogel passed through the catheter, the shearing rate increased to 1,140 s⁻¹. After the hydrogel exited a tip of the catheter, the shearing rate dropped sharply (to 0.01 s⁻¹). According to test results, Ca_{1.0}-Alg/MCG freshly prepared in Example 1 not only exhibited a shear-thinning behavior, but also could return to the original viscosity under the above shearing rate change. As shown in FIG. 13, a change of a storage modulus of Ca_{1.0}-Alg/MCG freshly prepared in Example 1 over time was tested. This storage modulus began to increase at around 4 min and reached a plateau at around 80 min. The storage modulus of Example 1 increases over time and can provide clinicians with sufficient operative time. As shown in FIG. 14, a change of a cumulative released quantity of calcium ions over time was tested. The cumulative released quantity of calcium ions gradually increased over time, and reached approximately 90% at around 80 min. This indirectly confirms the time-dependent modulus variation characteristic of the hydrogel in Example 1. As shown in FIG. 15, maximum pressures that could be withstood by the hydrogels in Comparative Example 1 and Example 1 were tested. An embolization pressure of MCG was approximately 12.1±1.6 kPa. An embolization pressure of Ca_{1.0}-Alg/MCG increased from the initial 22.2±1.2 kPa to 69.9±6.0 kPa. This confirms that the mechanical properties of Example 1 can be improved over time.

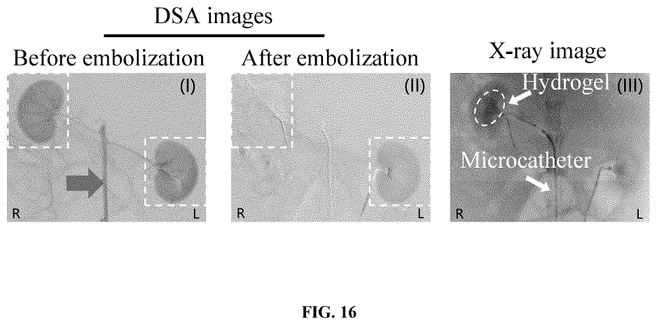

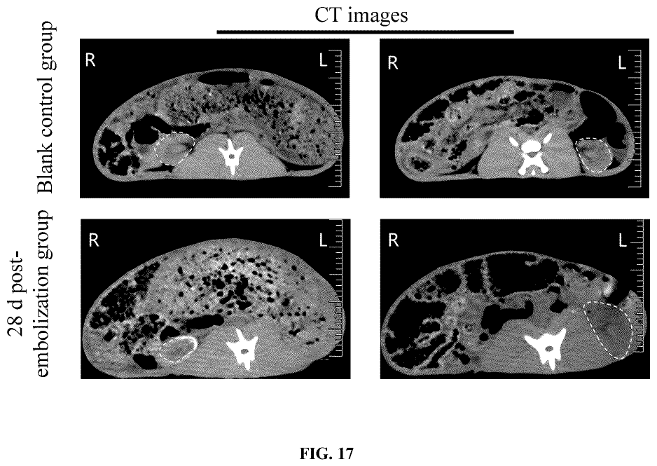

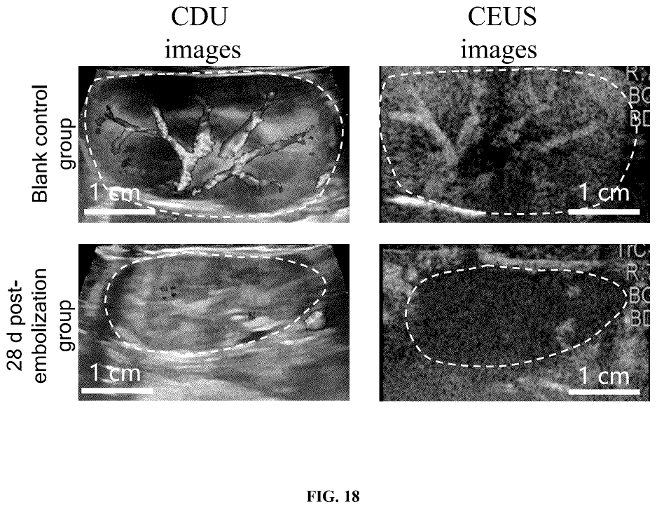

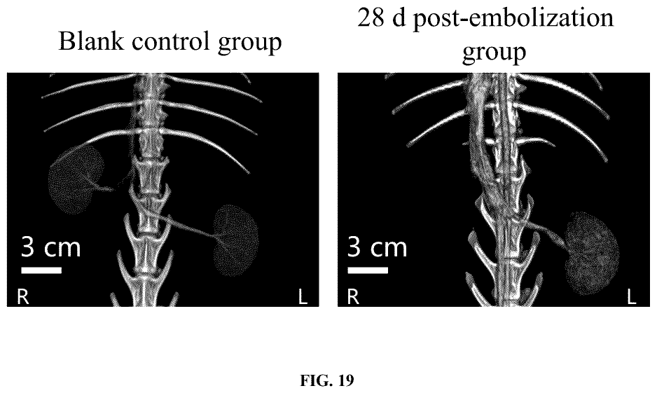

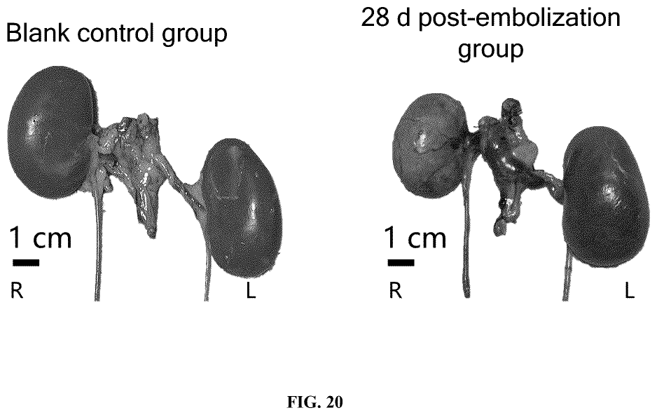

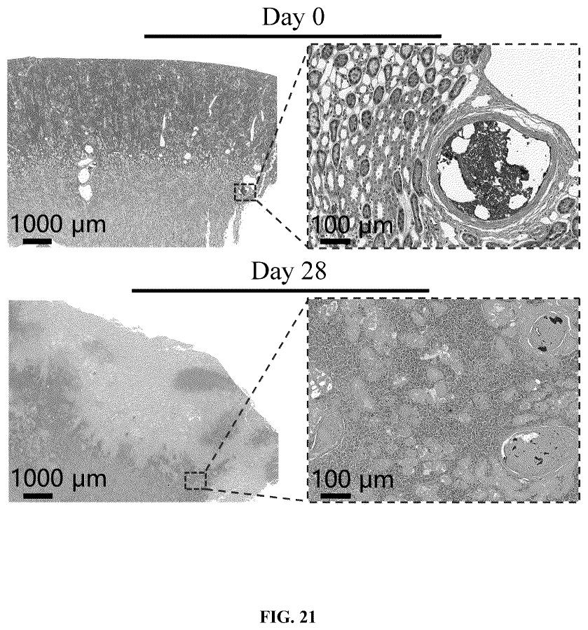

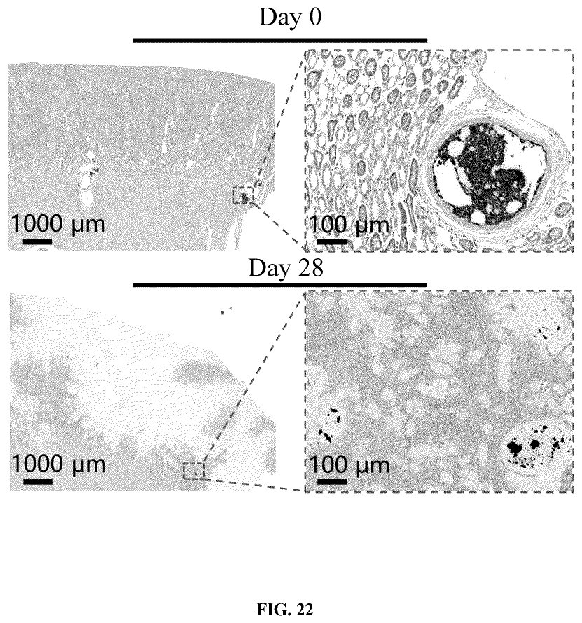

A rabbit renal embolization model was employed to evaluate the embolization efficacy of Example 1. As shown in FIG. 16, before embolization, DSA images revealed clear contours of double kidneys and the trajectory of blood vessels. The hydrogel freshly prepared in Example 1 was mixed with iodixanol, and then injected through a catheter into a right renal artery. According to X-ray imaging data, the hydrogel accumulated in renal arteries and fine blood vessels. A post-embolization DSA image revealed a clear contour of a left kidney, but did not reveal a vasculature of a right kidney, indicating complete embolization of the right kidney. As shown in FIG. 17, CT imaging data on day 28 after embolization revealed a significant reduction in a volume of the right kidney. FIG. 18 shows CDU and CEUS imaging data on day 28 after embolization. According to results, a kidney volume decreased and there was no blood supply on day 28 after embolization. According to three-dimensional reconstruction imaging data in FIG. 19, neither the right renal artery nor the right kidney was visible on day 28 after embolization. FIG. 20 shows pictures of kidneys on day 28 after embolization, which similarly confirm the atrophy of the right kidney. It can be seen that the hydrogel in Example 1 exhibits a prominent embolization effect. According to H&E staining images of renal tissues shown in FIG. 21, on day 28 after embolization, a cell nucleus of a renal tissue experienced extensive deformation and apoptosis, and the renal tissue underwent widespread necrosis. FIG. 22 shows PB staining results of renal tissues. These PB staining results confirm that the hydrogel can retain in a renal vasculature for a long time and can achieve stable and effective vascular embolization.

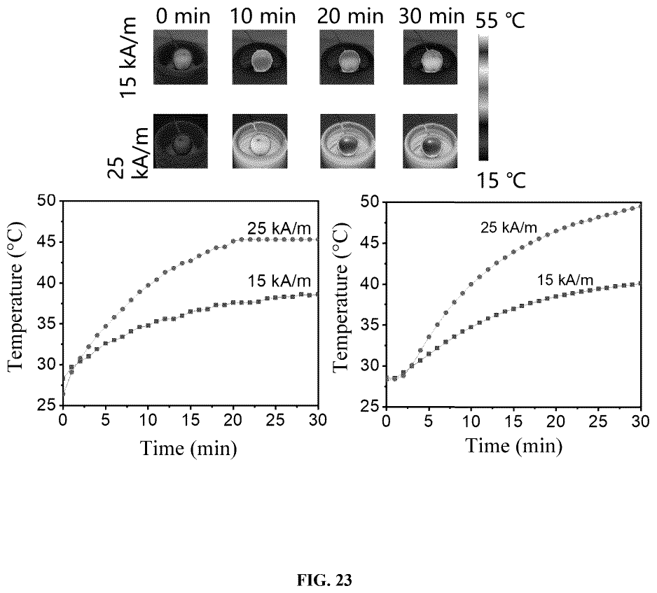

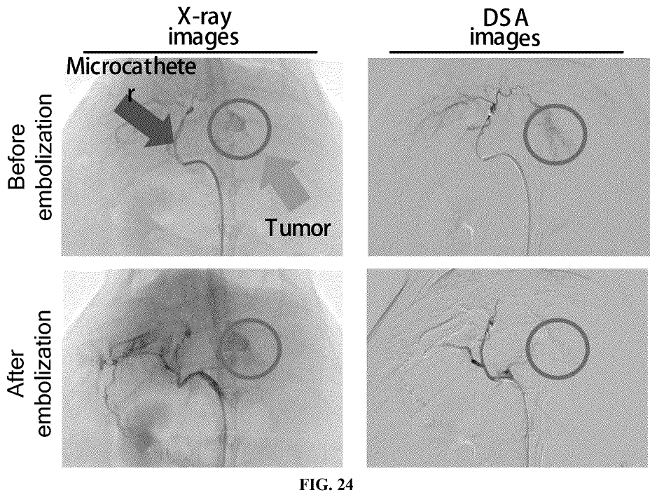

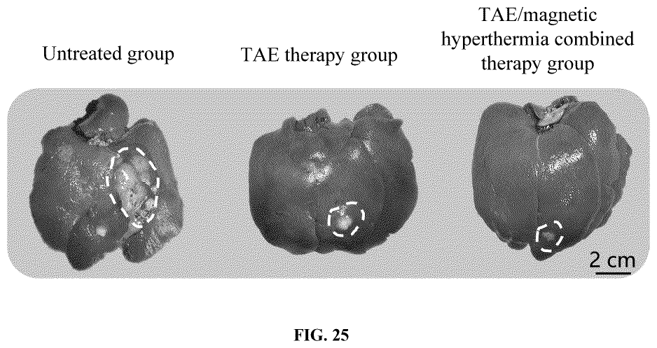

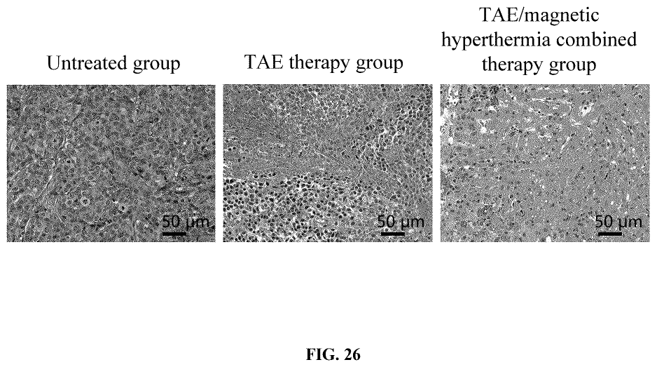

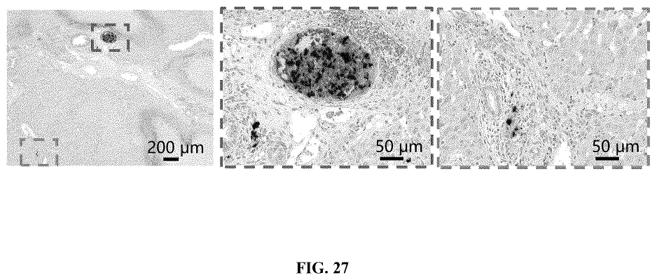

Due to improved magnetic responsiveness of iron oxide nanoparticles, whether a magnetothermal effect of the particle hydrogel with improved mechanical properties under an alternating magnetic field can kill tumor margin cells was assessed in a tumor model. A surface temperature of the tumor model was monitored by an infrared camera. An internal temperature change in the tumor model was measured by a fiber-optic temperature sensor. As shown in FIG. 23, under a field intensity of 25 kA/m, the surface temperature of the tumor model reached 45° C. at about 20 min. It confirms that the hydrogel can kill tumor margin cells under an alternating magnetic field. Embolization and magnetic hyperthermia effects of the hydrogel in Example 1 were validated in liver cancer rabbits. As shown in FIG. 24, a tumor location (marked by a green circle) and a tumor-feeding artery were visible before embolization. After embolization, the tumor location was invisible, confirming that a tumor vasculature had been embolized. FIG. 25 shows pictures of livers from an untreated group, a TAE therapy group, and a TAE/magnetic hyperthermia combined therapy group on day 14. According to the results, a tumor volume in the untreated group was significantly large. After TAE, a tumor volume decreased, indicating that embolization could inhibit tumor progression. The TAE/magnetic hyperthermia combined therapy group had a minimum tumor volume. It indicated accordingly that magnetic hyperthermia could further suppress tumor growth on the basis of embolization. According to H&E staining images of tumor tissues in FIG. 26, an increased number of tumor cell nuclei underwent deformation and apoptosis and there was extensive tissue necrosis in the TAE/magnetic hyperthermia combined therapy group compared to the TAE therapy group. It revealed that magnetic hyperthermia could significantly enhance the therapeutic effect of embolization. FIG. 27 shows PB staining images of tumor tissues. These results indicate that the hydrogel can achieve long-term vascular occlusion. The above experimental results prove that the hydrogel in Example 1 demonstrates stable and long-lasting embolization efficacy in blood vessels of tumors in liver cancer rabbits, and can be used for embolization/magnetic hyperthermia combined therapy to improve therapeutic outcomes.

The above are merely preferred examples of the present disclosure, and are merely illustrative rather than restrictive. It should be understood by those skilled in the art that many alterations, modifications, or even equivalent replacements can be made within the spirit and scope defined by the claims of the present disclosure, but such alterations, modifications, or equivalent replacements fall within the protection scope of the present disclosure.

What is claimed is:

1. A preparation method of a particle hydrogel with improved mechanical properties, comprising following steps:
S1, mixing a gelatin nanoparticle, an iron oxide nanoparticle, a calcium carbonate nanoparticle, and sodium alginate under alkaline conditions; and
S2, adding a glucono-δ-lactone (GDL) powder for pH regulation to produce the particle hydrogel with the improved mechanical properties,
wherein a preparation process of the gelatin nanoparticle in the step S1 comprises following steps:
S111, dissolving a gelatin in deionized water under heating to produce a gelatin solution; adding acetone to the gelatin solution, and allowing to stand at room temperature for 1 h to produce a supernatant and a precipitate; discarding the supernatant; and dissolving the precipitate in deionized water, and conducting lyophilization to produce a lyophilized high-molecular-weight gelatin; and
S112, dissolving the lyophilized high-molecular-weight gelatin obtained in the S111 in deionized water, and adjusting a pH with dilute hydrochloric acid to 2.5; under stirring, adding acetone dropwise to produce a gelatin dispersion; adding a glutaraldehyde solution to the gelatin dispersion at room temperature, and stirring for 16 h in the dark; adding an aqueous glycine solution, and stirring for 1 h; and conducting filtration and centrifugal washing to produce the gelatin nanoparticle; and
a preparation process of the iron oxide nanoparticle in the step S1 comprises following steps:
S121, adding 1.08 g of ferric chloride hexahydrate to a mixture of 10 mL of ethylene glycol and 30 mL of diethylene glycol, and stirring for 30 min to produce a first mixed solution;
S122, adding 0.1 g of polyacrylic acid to the first mixed solution obtained in the S121, and stirring for 30 min to produce a second mixed solution;
S123, adding 6 g of anhydrous sodium acetate to the second mixed solution obtained in the S122, and stirring for 1 h to produce a third mixed solution; and
S124, adding the third mixed solution obtained in the S123 to a high-pressure reactor, and conducting a reaction at a high temperature to produce a black product; and subjecting the black product to centrifugal washing to produce the iron oxide nanoparticle,
wherein the reaction in the S124 is conducted at 200° C. for 12 h.

2. The preparation method according to claim 1, wherein a preparation process of the calcium carbonate nanoparticle in the step S1 comprises following steps:
S131, dissolving 150 mg of calcium chloride dihydrate in 100 mL of ethanol to produce a calcium chloride solution;
S132, placing 5 g of an ammonium bicarbonate powder in a sealed container; and
S133, adding the calcium chloride solution obtained in the S131 to the sealed container in the S132; placing the sealed container in an oven, and conducting a reaction under vacuum to produce a white product; and subjecting the white product to centrifugal washing to produce the calcium carbonate nanoparticle.

3. The preparation method according to claim 2, wherein the reaction in the S133 is conducted at 40° C. for 24 h.

4. The preparation method according to claim 1, wherein the gelatin nanoparticle, the iron oxide nanoparticle, the calcium carbonate nanoparticle, and the sodium alginate in the step S1 are mixed under vortexing at a pH of 11.

5. The preparation method according to claim 1, wherein in the step S2, a solid content in the particle hydrogel with improved mechanical properties is 11.0 w/v % to 13.5 w/v %.
